(19) United States
(12) Patent Application Publication
(10) Pub. No.: US 2007/0276214 A1
(43) Pub. Date: Nov. 29, 2007

(54) Systems and Methods for Automated Segmentation, Visualization and Analysis of Medical Images

(76) Inventors: Frank C. Dachille, Amityville, NY (US); Dongqing Chen, Setauket, NY (US); Michael Meissner, Minneapolis, MN (US); Wenli Cai, Dorchester, MA (US)

Correspondence Address: Frank Chau, Esq.;F. Chau & Associates, 130 Woodbury Road, Woodbury, NY, 11797, US

(21) Appl. No.: 10/580,763

(22) PCT Filed: Nov. 26, 2004
(86) PCT No.: PCT/US04/39747

§ 371 Date: Apr. 16, 2007

Related U.S. Application Data
(60) Provisional application No. 60/525,603, filed on Nov. 26, 2003.
(60) Provisional application No. 60/617,559, filed on Oct. 9, 2004.

(51) Int. Cl. A61B 5/00

(52) U.S. Cl. 600/407

(57) ABSTRACT

An imaging system for automated segmentation and visualization of medical images (100) includes an image processing module (107) for automatically processing image data using a set of directives (109) to identify a target object in the image data and process the image data according to a specified protocol, a rendering module (105) for automatically generating one or more images of the target object based on one or more of the directives (109) and a digital archive (110) for storing the one or more generated images. The image data may be DICOM-formatted image data (103), wherein the imaging processing module (107) extracts and processes meta-data in DICOM fields of the image data to identify the target object. The image processing module (107) directs a segmentation module (108) to segment the target object using processing parameters specified by one or more of the directives (109).

### CROSS-REFERENCE TO RELATED APPLICATIONS

This application claims priority to U.S. Provisional Application No. 60/525,603, filed Nov. 26, 2003, and U.S. Provisional Application No. 60/617,559, filed on Oct. 9, 2004, which are fully incorporated herein by reference.

### TECHNICAL FIELD OF THE INVENTION

The present invention relates generally to systems and methods for aiding in medical diagnosis and evaluation of internal organs (e.g., blood vessels, colon, heart, etc.) More specifically, the invention relates to a 3D visualization system and method for assisting in medical diagnosis and evaluation of internal organs by enabling visualization and navigation of complex 2D or 3D data models of internal organs, and other components, which models are generated from 2D image datasets produced by a medical imaging acquisition device (e.g., CT, MRI, etc.).

### BACKGROUND

Various systems and methods have been developed to enable two-dimensional (“2D”) visualization of human organs and other components by radiologists and physicians for diagnosis and formulation of treatment strategies. Such systems and methods include, for example, x-ray CT (Computed Tomography), MRI (Magnetic Resonance Imaging), ultrasound, PET (Positron Emission Tomography) and SPECT (Single Photon Emission Computed Tomography).

Radiologists and other specialists have historically been trained to analyze scan data consisting of two-dimensional slices. Three-Dimensional (3D) data can be derived from a series of 2D views taken from different angles or positions. These views are sometimes referred to as “slices” of the actual three-dimensional volume. Experienced radiologists and similarly trained personnel can often mentally correlate a series of 2D images derived from these data slices to obtain useful 3D information. However, while stacks of such slices may be useful for analysis, they do not provide an efficient or intuitive means to navigate through a virtual organ, especially one as tortuous and complex as the colon, or arteries. Indeed, there are many applications in which depth or 3D information is useful for diagnosis and formulation of treatment strategies. For example, when imaging blood vessels, cross-sections merely show slices through vessels, making it difficult to diagnose stenosis or other abnormalities.

### SUMMARY OF THE INVENTION

The present invention is directed to systems and methods for visualization and navigation of complex 2D or 3D data models of internal organs, and other components, which models are generated from 2D image datasets produced by a medical imaging acquisition device (e.g., CT, MRI, etc.). In one exemplary embodiment, an imaging system for automated segmentation and visualization of medical images includes an image processing module for automatically processing image data using a set of directives to identify a target object in the image data and process the image data according to a specified protocol, a rendering module for automatically generating one or more images of the target object based on one or more of the directives and a digital archive for storing the one or more generated images. The image data may be DICOM-formatted image data, wherein the imaging processing module extracts and processes meta-data in DICOM fields of the image data to identify the target object. The image processing module directs a segmentation module to segment the target object using processing parameters specified by one or more of the directives.

These and other exemplary embodiments, aspects, features and advantages of the present invention will become apparent from the following detailed description of preferred embodiments, which is to be read in connection with the accompanying drawings.

### BRIEF DESCRIPTION OF THE DRAWINGS

FIG. 1 is a diagram of a 3D imaging system according to an embodiment of the invention.

FIG. 2 is a flow diagram illustrates a method for automatic processing of medical images according to an exemplary embodiment of the invention.

FIG. 3 is a flow diagram illustrating method for heart segmentation according to an exemplary embodiment of the invention

FIGS. 4A and 4B are exemplary images of a heart, which schematically illustrate the heart segmentation method of FIG. 3.

FIG. 5 is an exemplary curved MPR image illustrating display of blood lumen information graphs along a selected vessel on the curved MPR image according to an exemplary embodiment of the invention.

### DETAILED DESCRIPTION OF EXEMPLARY EMBODIMENTS

The present invention is directed to medical imaging systems and methods for assisting in medical diagnosis and evaluation of a patient. Imaging systems and methods according to preferred embodiments of the invention enable visualization and navigation of complex 2D and 3D models of internal organs, and other components, which are generated from 2D image datasets generated by a medical imaging acquisition device (e.g., MRI, CT, etc.).

It is to be understood that the systems and methods described herein in accordance with the present invention may be implemented in various forms of hardware, software, firmware, special purpose processors, or a combination thereof. Preferably, the present invention is implemented in software as an application comprising program instructions that are tangibly embodied on one or more program storage devices (e.g., magnetic floppy disk, RAM, CD ROM, DVD ROM, ROM and flash memory), and executable by any device or machine comprising suitable architecture.

It is to be further understood that since the constituent system modules and method steps depicted in the accompanying Figures are preferably implemented in software, the actual connection between the system components (or the flow of the process steps) may differ depending upon the manner in which the present invention is programmed. Given the teachings herein, one of ordinary skill in the related art will be able to contemplate these and similar implementations or configurations of the present invention.

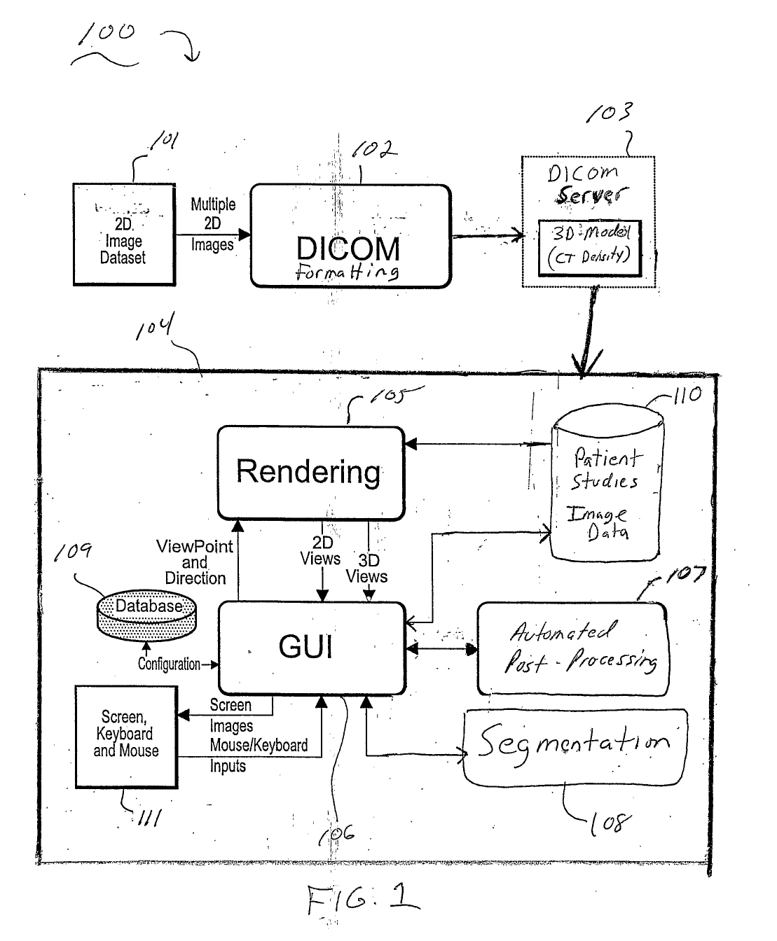

FIG. 1 is a diagram of an imaging system (100) according to an embodiment of the present invention. The imaging system (100) comprises an image acquisition device that generates 2D image datasets (101) which are formatted in DICOM format by DICOM module (102). For instance, the 2D image dataset (101) may comprise a CT (Computed Tomography) dataset (e.g., Electron-Beam Computed Tomography (EBCT), Multi-Slice Computed Tomography (MSCT), etc.), an MRI (Magnetic Resonance Imaging) dataset (12), an ultrasound dataset (13), a PET (Positron Tomography) dataset, an X-ray dataset and SPECT (Single Photon Emission Computed Tomography) dataset. A DICOM server (103) provides an interface to DICOM system (102) and receives and process the DICOM-formatted datasets received from the various medical image scanners. The server (103) may comprise software for converting the 2D DICOM-formatted datasets to a volume dataset. The DICOM server (103) can be configured to, e.g., continuously monitor a hospital network and seamlessly accept patient studies automatically into a system database the moment such studies are “pushed” from an imaging device.

The imaging system (100) further comprises a 3D imaging tool (104) that executes on a computer system. The imaging tool (104) comprises various modules including a rendering module (106), a user interface module (106) and automated post-processing module (107), a segmentation module (108), databases (109) and (11) and a plurality of I/O devices (111) (e.g., screen, keyboards, mouse, etc.). The 3D imaging tool (104) is a heterogeneous image-processing tool that is used for viewing selected anatomical organs to evaluate internal abnormalities. With the imaging tool (104), a user can display 2D images and construct a 3D model of various organs, e.g., vascular system, heart, colon, etc. In general, the UI (106) provides access points to menus, buttons, slider bars, checkboxes, views of the electronic model and 2D patient slices of the patient study. The user interface is interactive and mouse driven, although keyboard shortcuts are available to the user to issue computer commands. The 3D imaging tool (104) can receives the DICOM-formatted 2D images and 3D images via server (103) and generate 3D models from a CT volume dataset derived from the 2D slices using known techniques (wherein an original 3D image data set can be used for constructing a 3D volumetric model, which preferably comprises a 3D array of CT densities stored in a linear array).

The GUI module (106) receives input events (mouse clicks, keyboard inputs, etc.) to execute various functions such as interactive manipulation (e.g., artery selection, segmentation) of 3D models. The GUI module (106) receives and stores configuration data from database (109). The configuration data comprises meta-data for various patient studies to enable a stored patient study to be reviewed for reference and follow-up evaluation of patient response treatment. The database (109) further comprises initialization parameters (e.g., default or user preferences), which are accessed by the GUI (30) for performing various functions.

The rendering module (105) comprises one or more suitable 2D/3D renderer modules for providing different types of image rendering routines according to exemplary embodiments of the invention as described herein. The renderer modules (software components) offer classes for displays of orthographic MPR images and 3D images. The rendering module (105) provides 2D views and 3D views to the GUI module (106) which displays such views as images on a computer screen. The 2D views comprise representations of 2D planer views of the dataset including a transverse view (i.e., a 2D planar view aligned along the Z-axis of the volume (direction that scans are taken)), a sagittal view (i.e., a 2D planar view aligned along the Y-axis of the volume) and a Coronal view (i.e., a 2D planar view aligned along the X-axis of the volume). The 3D views represent 3D images of the dataset. Preferably, the 2D renderers provide adjustment of window/level, assignment of color components, scrolling, measurements, panning zooming, information display, and the ability to provide snapshots. Preferably, the 3D renderers provide rapid display of opaque and transparent endoluminal and exterior images, accurate measurements, interactive lighting, superimposed centerline display, superimposed locating information, and the ability to provide snapshots.

The rendering module (105) presents 3D views of 3D models (image data) that are stored in database (110) to the GUI module (106) based on the viewpoint and direction parameters (i.e., current viewing geometry used for 3D rendering) received from the GUI module (106). The 3D models stored in database (110) include original CT volume datasets and/or tagged volumes. A tagged volume is a volumetric dataset comprising a volume of segmentation tags that identify which voxels are assigned to which segmented components, or which are tagged with other data (e.g., vesselness for blood vessels) Preferably, the tag volumes contain an integer value for each voxel that is part of some known (segmented region) as generated by user interaction with a displayed 3D image (all voxels that are unknown are given a value of zero). When rendering an image, the rendering module (105) overlays an original volume dataset with a tag volume, for example.

The automated post-processing module (107) includes methods that enable automatic processing of medical images according to exemplary embodiments of the invention. More specifically, the automated post-processing module (107) comprises a plurality of methods to automatically process 2D or 3D image datasets to identify target organs of interest in the image datasets and generate images of such target organs without user intervention. As explained below, the automated post-processing module (107) uses a set of predefined rules (stored in configuration database (109) to process meta-data associated with the image dataset to automatically identify one or more organs of interest that are the subject of the image dataset and to automatically determine the processing protocol(s) to be used for processing the image dataset. Such processing protocols set forth the criteria and parameters that are used for automatically segmenting target organs of interest (via the segmentation module (108) and generating images of such segmented organs (via the rendering module (105).

The segmentation module (108) comprises methods that enable user interactive segmentation for classifying and labeling medical volumetric data, according to exemplary embodiments of the invention. The segmentation module (1080 comprises functions that allow the user to create, visualize and adjust the segmentation of any region within orthogonal, oblique, curved MPR slice image and 3D rendered images. The segmentation module (108) produces volume data to allow display of the segmentation results. The segmentation module (103) is interoperable with annotation methods to provide various measurements such as width, height, length volume, average, max, std deviation, etc of a segmented region. As explained below, the imaging tool (104) comprises methods that enable a user to set specific volume rendering parameters, perform 2D measurements of linear distances and volumes, including statistics (such as standard deviation) associated with the measurements, provide an accurate assessment of abnormalities; and enable synchronized views among different 2D and 3D models. Various features and functions of the exemplary imaging tool (104) will now be discussed.

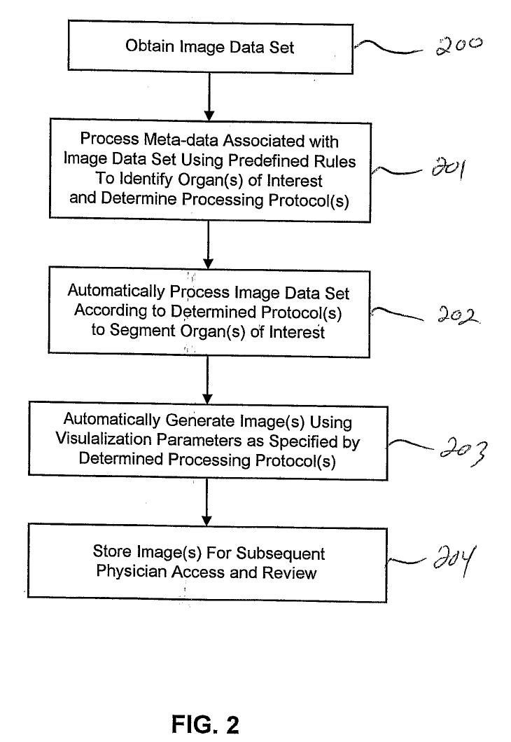

FIG. 2 is a flow diagram illustrates a method for automatic processing of medical images according to an exemplary embodiment of the invention. More specifically, FIG. 2 depicts a method for automatic selection of processing protocols for segmenting organs of interest and generating images for visualization of such organs. In general, the exemplary method of FIG. 2 is an automated procedure in which 2D or 3D image datasets are automatically processed to identify target organs of interest in the image datasets and generate images of such target organs without user intervention. In other words, the exemplary method provides an automated post-processing method which automatically processes acquired image data and reconstructs 2D or 3D for viewing without requiring user intervention during such post-processing.

More specifically, referring to FIG. 2, the exemplary process begins with obtaining an image data set (step 200). The image data set may comprise a sequence of adjacent 2D slices or a 3D volumetric data set comprising raw image data that is acquired via a body scan of an individual using one of various imaging modalities including, for example, CT, MRI, PET, US, etc.). Next, a set of predefined rules are used to process meta-data associated with the image dataset to automatically identify one or more organs of interest that are the subject of the image dataset and to automatically determine the processing protocol(s) to be used for processing the image dataset (step 201). The processing protocols set forth the criteria and parameters that are used for automatically segmenting target organs of interest and generating images of such segmented organs.

In one exemplary embodiment of the invention, the meta-data supplied as part of the scan procedure (scanner supplied DICOM data fields), which is included with the image dataset can be used to identify target organs of interest. Indeed, medical data is usually supplied in DICOM format which contains image data along with meta-data in the form of numerous textual fields that specify the purpose of the exam and content of the data, as well as provide other supplementary information (e.g., patient name, gender, scanning protocol, examining physician or health organization, etc.). For example, each hospital has its own specific way of filling out such DICOM text fields which helps to route the images and to aid in billing and diagnosis. In accordance with an exemplary embodiment of the invention, these data fields are interpreted using flexible, customizable rules, to provide appropriate processing based on the type of data received.

The predefined rules (user-defined/customizable, default rules) are used to determine the organ(s) of interest. The set of rules are processed in order until a true condition is met. By way of example, each rule allows some logical combination of tests using the DICOM field data with string matching or numerical computation and comparisons. Each rule also specifies a resulting “processing protocol” that permits improved processing of the identified organ(s) of interest (e.g., vessels, heart, etc. Thus, when the organ(s) of interest are identified, the image data set can be automatically processed to segment the organ(s) of interest and apply other processing methods according to the specified processing protocol (step 202). For example, the processing protocol would specify which regions of anatomy to focus on, what features to process, the range of CT values to focus processing on, and even allow for hiding part of the dataset from visibility during visualization to allow for better focusing of the radiologists attention on the important data. By way of example, based on body part, automatic body-part-specific segmentation and vessel enhancement can proceed using parameters that are tuned to improve the recognition and delineation of organs such as vessels, heart, etc. For example, if one is interested only in large vessels, then many of the small vessels can be ignored during the processing phase. This allows improvements in accuracy and speed—improving overall diagnosis time and quality.

The desired segmentation and visualization protocols can be automatically determined based on some information either within the image data (if it looks like a heart, then process it as using the heart protocol; if it looks like a lung, processes it using the lung protocol), meta-data attached to the image (e.g., using one of the ‘tag°fields in DICOM), or by user-input configuration at the computer console. In more detail, one possible mechanism by which to indicate the desired protocol is for the scanner operator to input the protocol to the scanner which encodes this information along with the image data after the scan is completed. Another mechanism is for a person to select on the computer the desired protocol from a list of available protocols. Another mechanism is for the computer to automatically determine the protocol using whatever information is available in the image data (if it looks like a heart, use the heart protocol, etc.) and the metadata that comes along with each image (e.g., the referring physician's name is “Jones” and he prefers protocol “A” for heart scans, except for short, female patients with heart scans on Tuesdays he prefers protocol “B”). As can be seen, the possibilities for automatic selection are virtually unlimited because the protocol can be derived from so many factors including the unique data scanned in every image.

For example, if the image data set is the we have a chest CT exam and we know that the reason for the scan is to examine the coronary arteries, then we can process just the coronary arteries and inhibit processing of the pulmonary (lung) vessels. This speeds up the process and lets the doctor focus on just the task at hand. The lung vessels can always be examined as well, but this would usually require a re-processing with a new specific emphasis placed on the lungs. (The user would select the dataset, right click, and select “re-process as lung case”, wait a few minutes, then open the case again to examine the lungs.) In this example, two choices for a chest CT scan would be (i) automatic segmentation of heart and lungs. For instance, segmentation of the heart can include removal of ribs, but leaving the spine and sternum for reference, wherein the ribs and lungs are hidden from view during visualization and excluded from processing for faster processing. Hiding the ribs from view allows the radiologist to easily see the heart from all directions during examination without having to see past or manually cut away the ribs to see the heart. Exemplary embodiments of the invention for segmenting the heart by removing ribs and lungs will be discussed below. Moreover, removing large blood pools from the heart region can prevent the left and right ventricles and atria from being effectively hidden. Indeed, when examining the coronary vessels, these structures interfere with visualization because they are large and bright (just like an outdoor floodlight makes it difficult to stargaze). Moreover, it may be desirable to enhance small vessels (about 2-5 mm in diameter) with high contrast to surrounding tissue and low average CT values. Enhancement is applied to not only straight vessels, but also those with a lot of small wiggles (high curvature) and branches.

Next, one or more images are automatically generated of the segmented organs of interest using visualization parameters as specified by the processing protocols (203). Visualization parameters can then be automatically selected. When viewing a dataset, there are a great number of parameters that need to be adjusted in order to obtain a useful diagnostic view. For instance, Hospital A may require that every time there is a brain study that the color scheme should be from blue to red to white and the contrast/brightness (called window/level by radiologists) should be set to 70/150 and the view should be a 3D view from top left, top right, top middle, and front and furthermore the vessels should be shown enhanced by 25% and a splash of purple color. On the other hand, Hospital B may desire a different set of images be created with all of the parameters different and even the view not 3D, but a sequence of 2D slices at some oblique (not parallel to any axis) angle. To satisfy all of these disparate possibilities, the entire set of visualization parameters can be encapsulated in a set of “visualization presets” which allows for automated generation of views and even automated post-processed images to be generated.

These visualization parameters may include:

(i) Selection of 3D viewpoints, which are designed to match standard hospital procedures such as cardiac, aortic, or brain catheterization., or other user-customizable set of viewpoints.

(ii) Selection of a set of aforementioned 3D viewpoints that are automatically captured and saved to digital or film media. Either the presets can be used as a starting point for interactive exploration or they may be used to generate a set of images automatically.

(iii) Selection of contrast/brightness setting or set of settings (called “window/level” in the parlance of radiology) specific to the body part

(iv) Selection of 3D opacity transfer function (or set of transfer functions) specific to the body part.

For every type of anatomy, there is usually as set of visualization techniques that are optimal for diagnosis. For viewing vessels, it is desirable to visualize the vessel in a curved MPR view and a rotating 3D view. For lung nodules, a “cartwheel” projection that shows a small square oblique MPR view that rotates 180 degrees around a central axis of a suspected lung tumor. For virtual colonoscopy, it is desirable to have a 3D flythrough along the entire length of the colon. Moreover, for viewing vessels, it may be desired to generate a set of images through the carotid artery, a branching structure that makes visible the three primary vessels at the bifurcation all in a single plane. There is one unique plane that passes through the three vessels and an MPR image can be aligned to the plane to image the vessels most clearly. That MPR image plane can be slid back and forth parallel to itself to generate a set of images that together cover the entire vascular structure. Another doctor may desire a set of images that takes the same three vessels, renders them using 3D volume rendering from the front side of the patient and rotates the object throughout 360 degrees around a vertical axis producing 36 color images at a specified resolution, one image every ten degrees. Still another doctor may desire to have each of the three vessels rendered independently using 3D MIP projection every 20 degrees, thereby producing three separate sets of images (movies) each with 18 frames.

After the images are automatically prepared, such images can be stored in any suitable electronic form (step 204). The general practice in modern radiology departments is for all digital images to be stored in a Picture Archiving and Communication System (PACS). Such a system centralizes and administrates the storage and retrieval of digital images from all parts of the hospital to every authorized user in a hospital network. It usually is a combination of short-term and long-term storage mechanisms that aim to provide reliability, redundancy, and efficiency. To read images within such a system, the radiologists usually selects a patient and the “series” or sets of images in the study are recalled from the PACS and made available for examination on a PACS viewer usually on a standard personal computer. As noted above, the images can be, for example, select 2D images from the original acquisition, 2D multi-planar reformatted (MPR) images either in an axis orthogonal to the original image plane or in any axis, curved MPR images in which all the scan lines are parallel to an arbitrary line and cut through a 3D curve, or 3D images using any projection scheme such as perspective, orthogonal, maximum intensity projection (MIP), minimum intensity projection, integral (summation), to name a few. Furthermore, fused or combined images from multiple modalities (CT, MRI, PET, ultrasound, etc.) using any of the image types mentioned above can be generated to add to the diagnostic value, once the anatomy has been matched between the separate acquisitions. In addition to the type of images desired, the appropriate number, size, color, quality, angle, speed, direction, thickness, and field of view of the images must also be selected. This choice varies significantly from doctor to doctor, but it is usually related or proportional to the size, shape, and/or configuration of the desired object(s).

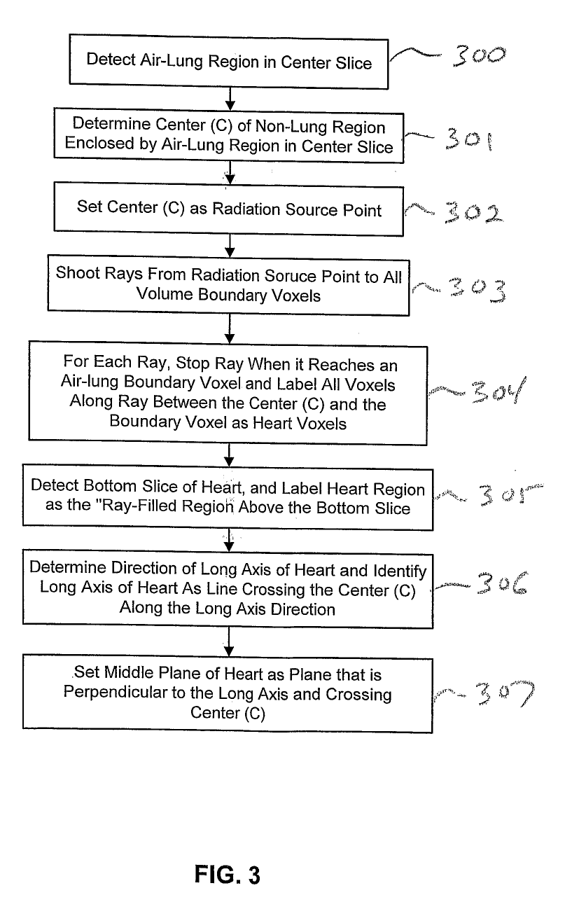

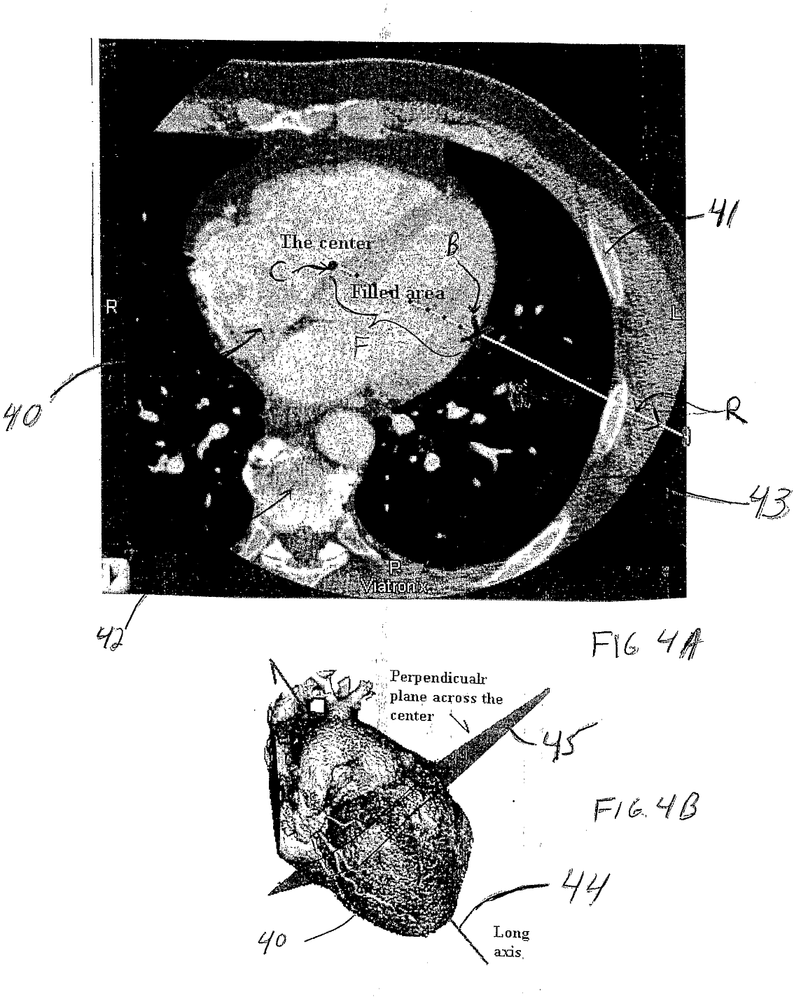

FIG. 3 is a flow diagram illustrating method for heart segmentation according to an exemplary embodiment of the invention. In particular, FIG. 3 depicts and exemplary method for heart segmentation which employs a “Radiation-Filling” method according to an exemplary embodiment of the invention. FIGS. 4A and 4B are illustrative views of a heart to schematically depict the exemplary method of FIG. 3. In general, the heart is enclosed by the lungs and ribs. FIG. 4A is an exemplary center slice of an axial image of a heart (40) showing ribs (41) and a spine/aorta region (42), wherein the air region of the lung is depicted in a darker-shaded color. The heart muscle and lumen have much brighter color than that of the air-filled lung. The heart is usually scanned from the top to the bottom and the scanning protocol often creates 200˜300 slice images with slice thickness of 0.5˜1 mm. The center slice is close to the middle axial plane of the heart.

Referring to FIG. 3, an initial step is to detect the air-lung region in a center slice of the heart (step 300). The lung region can be determined by simple threshholding technique. After the lung region has been extracted, a “radiation filling” process is applied to determine a region that is enclosed by the lung region. In one exemplary embodiment, this process involves determining the center (C) of the non-lung region that is enclosed by the air-lung region in the center slice (step 301) and the center (C) is set a “radiation source” point (step 302). Thereafter, a rays (R) is shot from the center C in all directions for purposes of determining the volume boundary voxels (step 303). For each ray that is shot, when the ray reaches an air-lung boundary voxel, all voxels along the ray between the center C and the boundary voxel are deemed heart voxels (step 305).

This step is depicted for example in FIG. 4A, wherein a ray R shot from the center C intersects a boundary voxel B between the heart region (40) and the air-lung region. Once the lung region is extracted, the “radiation filling” process is used to determine the region that is enclosed by the lung region. For the image volume, the voxel grid is in a finite setting. If a ray is shot for each voxel around the image volume boundary, the ray will cross over all voxels in the volume. Hence, shooting rays to all volume boundary voxels will enable the entire volume to be covered. By labeling all voxels along each ray between the center C and the corresponding boundary voxel, the region that is enclosed by the lung is delineated and this region contains heart (excluding the lung and ribs). The sternum and spine may be maintained in the image for an anatomical reference.

Referring again to FIG. 3, a bottom slice of the heart is detected and the heart region is defined as the “ray-filled region above the bottom slice (step 305). In one exemplary embodiment, the heart bottom slice can be determined by finding the lowest contrast-enhanced voxels. Next, the direction of the long axis of the heart is determined and the long axis is identified as a line that crosses the center C along the long axis direction (step 306). In one exemplary embodiment of the invention, the long axis direction is determined by applying a scattering analysis to the heart region and the direction of the maximum diverged direction is the determined as the direction of the long axis. Thereafter, the plane that is perpendicular to the long axis and crossing the center C is deemed the middle plane for the heart (step 307). This is depicted in the exemplary diagram of FIG. 4B, which illustrates the long axis (44) extending through the heart (40) and the center plane (45) which crosses the center C and is perpendicular to the long axis (44). The heart is an oval shape in 3D. As noted above, the long axis of the oval can be determined by finding the maximum scattering direction of the heart masses. This can be solved by employing the Principal Analysis to all coordinate vectors of heart region, which is known to those of ordinary skill in the art, and the principal analysis will determine the maximum scattering direction. The short axis is located at the plane that crosses the center of the heart and is perpendicular to the long axis.

In other exemplary embodiments of the invention, rendering methods are implemented which enable synchronization of views containing a specific annotation to enable a specific annotation is visible in all applicable views. An annotation is a user-selected measurement or text placement in the image, which is used to determine a specific quantity for some attribute of the data such as a length, area, angle, or volume or to draw attention to a particular feature (using an arrow or some text label). A measurement may make sense to visualize in more than one way or in more than one image. For example, a length may be seen in a standard cross-sectional image, in a 3D image, in an oblique MPR image, or in a curved MPR image. When one or more windows on the screen show parts of the data that may be manipulated in such a way as to show the annotation, it is useful for the view to automatically show the view that best exhibits the annotation. For example, if slice 12 of a given data set is currently displayed and there is an annotation that is on slice 33, the view should automatically jump to slice 33 when the user selects the annotation from a central list of annotations elsewhere in the user interface. By way of further example, if the annotation is a line segment that measures a length, and there is also a 3D view on the screen, it would be useful to show the 3D view from an angle that best exhibits the length (i.e., perpendicular to the viewing direction) and which is zoomed to see the length clearly (not overfilling or underfilling the image).

In other exemplary embodiments, user interface and rendering methods are implemented that enables a user to select and arbitrary plane for a double-oblique slice or slab view. For example, in one exemplary embodiment, starting with an axial, sagittal or coronal image of some anatomy, a user can draw a line across a desired region of the image (clicking and dragging a mouse cursor). For instance, the user may curt through the middle of some anatomical object to render an oblique view. The new plane of is created by extruding the line into the image (i.e., the line can be viewed as the edge of the plane). A new view will then be rendered for the new plane and displayed to the user.

Moreover, methods are implemented to enable user-adjustment of a double-oblique view (arbitrary plane) by tilting the plane about the center of the image in any arbitrary direction. A double-oblique view is a plane that is not perpendicular to any of the primary image axes. Such view can be generated by starting with a standard cross-sectional view perpendicular to the Z-axis, then rotating the view plane about the X and/or Y axis by an angle which is not a multiple of 90 degrees. The double-oblique view enables visualization of a human anatomy that is not disposed in a perfect X, Y, or Z plane, but oriented at some other arbitrary angle.

More specifically, in one exemplary embodiment, adjustment (tilting) of the plane is performed about a set of known axes (e.g., horizontal, vertical, diagonal, or about image perpendicular axis). The tilting can be performed by rotating the plane as one would rotate a 3D image, e.g., by clicking and dragging an object in the image in the direction of a desired rotation. By way of example, in the case of a Z-slice that is desired to tilt about the vertical axis (in the current view), the user can select (via mouse click) the center of the image and then drag the center to the right or left. To simultaneously tilt the plane about the vertical and horizontal view axes, the mouse can be clicked in the center and dragged toward the upper right of the image to effect a tilting in that direction. Alternatively, special keys or GUI elements can be used to tilt the view in common directions. Furthermore, translation of the center of the view (often called panning) can be performed by clicking the mouse somewhere on the image and dragging it in the direction of the desired translation.

In other exemplary embodiments of the invention, a vessel segmentation and visualization system according to the invention enables selection and storage of multiple blood vessels for rapid reviewing at a subsequent time. For instance, a plurality of blood vessels that have been previously segmented, processed, annotated, etc. can be stored and later reviewed by selecting them one after another for rapid review. By way of further example, a plurality of different views may be simultaneously displayed in different windows (e.g., curved MPR, endoluminal view, etc.) for reviewing a selected blood vessel. When a user selects another stored (and previously processed) blood vessel, all of the different views can be updated to include an image and relevant information associated with the newly selected blood vessel. In this manner, a user can select one or more multiple views that the user typically uses for reviewing blood vessels, for instance, and then selectively scroll through some or all of the stored blood vessels to have each of the views instantly updated with the selected blood vessels to rapidly review such stored set of vessels.

For example, a typical view contains a set of images that show different aspects of a particular vessel (e.g., an overview, a curved MPR view, and a detail view such as an endoluminal or cross-sectional view, and also an information view with various quantities). Typically, a user will select a vessel with some picking mechanism, and then analyze the vessel in detail using the views. Then, to analyze another vessel, the user will clear the current vessel and repeat the process for another vessel. The problem is that the vessel selection process can be time-consuming and a lower-paid worker can perform the task as easily as a highly-paid radiologist. Therefore, it is helpful to allow the lower-paid worker to select many vessels one after another, store all the information in the computer, and then have the highly-paid radiologist open the study along with all of the pre-selected vessel information. The radiologist can then select each of the vessels from a simple list and have all the views update with the current vessel visualization and information.

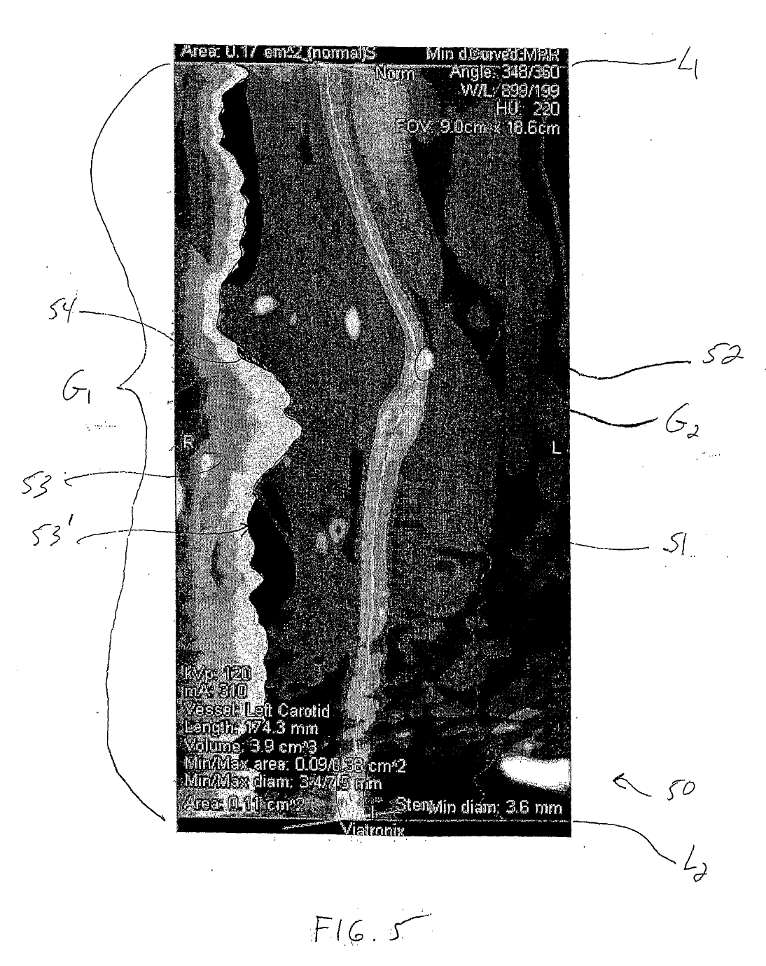

In other exemplary embodiments of the invention, vascular visualization methods are provided to enable display of blood lumen information graphs along a selected vessel on curved MPR and luminal MPR views. For instance, FIG. 5 is an exemplary curved MPR image (50) of a blood vessel (51) having a calcification area (52) (or hard plaque) on the lumen wall. The exemplary image (50) comprises a stacked graph (GI) displayed (e.g., superimpose) on the left side therefore. The stacked graph (G1) displays the lumen area (53) (enclosed by line 53′) of the vessel (51) along the length of the vessel between and bottom and top line (L1, L2). In addition, the stacked graph (G1) displays the calcification area (53) on top of the lumen area (53). In other words, in the exemplary embodiment of FIG. 5, the stacked graph (G1) illustrates total lumen area (53) with and depicts the area of the calcification (54), and the two quantities are shown as a stacked graph. Moreover, the exemplary image (50) further depicts a second graph G2 that graphically depicts a minimum diameter along the vessel (51) between lines L1 and L2. The lines L1 and L2 can be dragged by operation of a mouse to limit expand or contract the field of consideration.

In one exemplary embodiment, the lumen area (53) and calcification area (54) of the stacked graph can be displayed as different colors for ease of distinction. Moreover, other classifications/quantities can be included to provide a further breakdown of the composition of the vessel such as soft plaque, vulnerable plaque, etc. The composition can also be shown as a full grayscale distribution of the composition of the data in the vessel area. In particular, instead of showing one, two, or three bands of color that have a height corresponding to the area, all the voxels in the vessel area can be sorted and displayed lined up with the same coloring as the curved MPR view. Thus, such it shows at a glance the composition, size, and distribution of intensities within the vessel at every section along the length. This can be though of as a generalization of the two or three band composition discussed above, but carried out to N different bands of composition. So, it is a stacked graph with an infinite number of narrow bands plus the color coding of each band is the same as it is shown in the curved MPR or luminal MPR view.

In addition to those parameters/compositions shown above, other varying parameters can be displayed in graphical form synchronized alongside the vessel data, including, for example, estimated vessel stiffness; hemodynamic shear stress; hemodynamic pressure; presence of a molecular imaging contrast agent (one that visually tags soft plaque for example) estimated abnormalities (such as area discontinuities, aneurysms, dissections).

In other exemplary embodiments of the invention, visualization tools are provided to enable easy selection, segmentation and labeling of organ of interest such as vessels. For instance, exemplary embodiment of the invention include simplified segmentation methods that enable a user to readily segments vessels of interest including small coronary arteries to entire vascular systems. In general, a segmentation tool is provided which enables a user to place a seed point at a desirable voxel location, computing some similarity or desirability measure based on nearby (or global) information around the selected location, and allow the user to interactively grow parts of the dataset that are similar to the selected location and nearby. The exemplary segmentation tool allows direct selection of entire vascular structures. It can be difficult to specify a fixed threshold for selecting a desired structure in a medical dataset because of the noise and randomness of real data. Therefore, exemplary embodiment of the invention enable a user to select a small part of some object and interactively select more and more of the object until the desired amount is select or the selection process goes into an undesirable area.

An interactive segmentation method according to an exemplary embodiment of the invention will now be described in further detail. A user will enter a selection mode using any suitable command. The user will then select one or more parts of a desired object (it is not known as an object just yet by the computer, just a seed point or points). The user will drag the mouse cursor or some other GUI element to select the desired amount of growth from the seed point(s). The method responds to the GUI selection and shows a preview of the result of the growth. The user can continue to drag the mouse or GUI element to hone the selection area selecting either more or less area until satisfied. Once the selection is finalized, the user will exit the selection mode. With this method, interactive segmentation allow selection of more and less of the desired part based on a slider concept using distance along some scale as a metric to determine how much to include. The user can easily select the amount of segmentation by click of a mouse, for example. For instance, instead of varying a threshold value, an interactive segmentation method varies the number of voxels (i.e., the volume) of the desired object linearly, logarithmically, or exponentially in response to the slider input. This is in contrast to conventional methods in which the threshold (Hounsfield Units or HU) is varied. Indeed, varying the threshold can suddenly cause millions of voxels to be included with only a single value change in threshold depending on the data set.

A heap data structure (an ordered queue) can be used to determine which voxel to select next. As each voxel is selected, a list of neighbor voxels is placed into the queue, ordered by a measure of desirability. The desirability calculation is arbitrary and can be adjusted to suit the particular application. With an exemplary segmentation process, each preview of the selection can be shown in all applicable views. Moreover, the user can add a current selection to already existing selections.

The determination of desirability for intensity data can be in proportion to the absolute difference relative to the intensity at the seed point. For example, if the user clicks on a voxel with a value of 5, the user will assign a higher desirability to voxels that have values near 5 such as 4 and 6 and a low desirability to voxels such as 2 and 87. The determination of desirability can be in proportion to the vessel probability measure. In this case, it would be preferable to include voxels that have a higher probability of being a vessel (e.g., a higher vesselness value). In this case, the vesselness value is not compared to the seed point vesselness value, but instead the absolute quantity is used as a proportion to the desirability.

In other exemplary embodiments, the determination of desirability can be in negative proportion to the vessel probability measure (helpful for selecting non-vessel structures). The determination of desirability can be in proportion to a texture similarity measurement (e.g., using vector quantization of texture characteristics). The determination of desirability can be in proportion to shape-based similarity measurements (e.g., using curvature or n^{th} derivatives of the intensity, or other types of shape filters such as spherical or linear detection filters). The determination of desirability can be in proportion to some linear or non-linear combination of the above characteristics.

In other exemplary embodiments of the invention, when viewing 3D or 2D slabs images, various methods are implemented to increase the accuracy of user selections of components and objects, e.g., for curved path generation, seed point selection, or vessel endpoint selection, or 2D/3D localization. In general, when a user clicks on an image, the selected point is determined by the selection of the point along a 3D line which is defined by the click point extruded into the image. In one exemplary embodiment, the selected point is determined as the first point of intersection with the 3D object in which the voxel opacity or accumulated opacity reaches a certain threshold. In this exemplary embodiment, the concepts of volume rendering are implemented (e.g., the accumulation of opacity by a simulated light ray as it encounters voxels in the data set). This is in contrast to the typical method by which a ray is cast and the first voxel (or resampled field value) that is above a given threshold is used as the selection point. It is difficult to specify a fixed threshold that works well in all cases. Instead, the current visualization parameters that map voxels to opacity are used to determine the most likely desired selection. The idea is that the user has already adjusted the brightness/contrast and opacity ramp for the data as part of the general examination. Only then does the user want to select particular objects for more detailed examination. So, at this time, the light rays simulated by volume rendering are already stopping at the 50% ray opacity point on average. (Once a simulated light ray reaches 50% opacity, half of the photons that travel along that path are absorbed.) This is the median location for the photons to stop and the most probable location for the user to “see” when viewing a volume rendered image. With volume rendering, the accumulated effect of many different voxels along the light path is seen, but the user perceives the location at the median point of light absorption. This idea is now used to select the optimal pick point. A lower or higher value can also be used to provide an earlier or later pick point along the ray.

In another exemplary embodiment, the middle point of entrance and exit of the 3D object as determined by a voxel opacity threshold is determined as the selected (clicked point). When the user is selecting “objects” in a data set, the objects are often bounded on either side by non-visible regions (e.g., vessels are often surrounded by fat and bones are often surrounded by muscle). Once the user has adjusted the brightness/contrast and opacity color ramp and also selected the visibility of other selected objects in the data set, the desired object is often visible with non-opaque areas surrounding it. To conveniently pick the middle of these objects rather than the edge of those objects, a ray is cast along the click point in 3D, sample the data and convert to opacity along the ray, determine the entrance and exit points as determined by an opacity threshold, and select the middle between the points as the selection point.

In other exemplary embodiments of the invention, a tool is provided that enables a user to select from a single view an area based on a single seed point deposit and to automatically compute the perimeter of the object and other particulars such as minimum diameter, maximum diameter, etc. This feature is useful for determining various information about an object that is clearly differentiated from the surrounding tissue (e.g., tumor, calcification, nodule, polyp, etc.). With just a single selection, all the typical measurements and statistics can be computed and displayed to the user.

More specifically, with the included area of the object determined by an automatically derived threshold range, a sample of data surrounding the selection point can be used to automatically determine a threshold range that captures the majority of the object that shares similar characteristics. Hole-filling morphological operations can be used to simplify the edges of the object. Further, with the included area of the object determined by a similarity measure of intensity, texture, connectivity, and derivatives of the intensity, the intensity and some combination of the derived features can be used to automatically determine the boundary of the object. This can again be followed by hole-filling morphological operations. Also, the act of selection creates a set of annotations that describe the key characteristics of the area automatically and displays these to the user. The advantage is that the standard key measurements (such as the maximum and minimum diameter, volume, etc) can be generated automatically without extra manual steps.

Although the illustrative embodiments have been described herein with reference to the accompanying drawings, it is to be understood that the invention described herein is not limited to those precise embodiments, and that various other changes and modifications may be affected therein by one skilled in the art without departing from the scope or spirit of the invention. All such changes and modifications are intended to be included within the scope of the invention as defined by the appended claims.

What is claimed is:

1. A method for processing image data, comprising:
obtaining image data;
automatically processing the image data using a set of directives to identify a target object in the image data and process the image data according to a specified protocol; and
automatically generating one or more images of the target object based on one or more of the directives; and
storing the one or more generated images in a digital archive.

2. The method of claim 1, wherein the image data comprises DICOM-formatted image data.

3. The method of claim 2, wherein automatically processing the image data using a set of directives comprises processing meta-data in DICOM fields to identify the target object.

4. The method of claim 1, wherein automatically processing the image data comprises segmenting the target object using processing parameters specified by one or more of the directives.

5. An imaging system, comprising:
an image processing module for automatically processing image data using a set of directives to identify a target object in the image data and process the image data according to a specified protocol; and
a rendering module for automatically generating one or more images of the target object based on one or more of the directives; and
a digital archive for storing the one or more generated images.

6. The system of claim 5, wherein the image data comprises DICOM-formatted image data.

7. The system of claim 6, wherein the imaging processing module extracts and processes meta-data in DICOM fields of the image data to identify the target object.

8. The system of claim 5, wherein the image processing module directs a segmentation module to segment the target object using processing parameters specified by one or more of the directives.
